(19) United States
(12) Patent Application Publication
(10) Pub. No.: US 2024/0008730 A1
(43) Pub. Date: Jan. 11, 2024

(54) HYBRID WATER BLADDER FOR AN ENDOSCOPE

(71) Applicant: Boston Scientific Scimed, Inc., Maple Grove, MN (US)

(72) Inventors: Ryan V. Wales, Northborough, MA (US); Ryan Vincent William Pollock, Leominster, MA (US); John B. Golden, Norton, MA (US); Kurt Nicholas Robakiewicz, Upton, MA (US); Jeff Gray, Sudbury, MA (US); Paul Smith, Smithfield, RI (US)

(73) Assignee: Boston Scientific Scimed, Inc., Maple Grove, MN (US)

(21) Appl. No.: 18/348,977

(22) Filed: Jul. 7, 2023

Related U.S. Application Data
(60) Provisional application No. 63/359,747, filed on Jul. 8, 2022.

(51) Int. Cl. A61B 1/12

(52) U.S. Cl. CPC A61B 1/126

(57) ABSTRACT

Methods and systems for refilling a container during an endoscopic procedure. An illustrative container may comprise a container configured to contain a fluid within a first receptacle thereof, the container movable between a collapsed storage configuration and an expanded use configuration, a lens water supply tube including a first lumen extending therethrough, the first lumen is in fluid communication with the first receptacle of the container, a gas supply tube including a second lumen extending therethrough, the second lumen is in operative fluid communication with the first receptacle, and a port positioned adjacent to the top portion of the container. The port may be configured to be in selective fluid communication with the first receptacle of the container.

### CROSS-REFERENCE TO RELATED APPLICATIONS

This application claims the benefit of U.S. Prov. Pat. App. No. 63/359,747, filed Jul. 8, 2022, titled HYBRID WATER BLADDER FOR AN ENDOSCOPE, which is incorporated herein by reference.

### FIELD

This disclosure relates generally to medical fluid containers and tubing assemblies and methods, and particularly to a container and tube assembly to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these capabilities compressed gasses from either the processor or alternative source are used to increase the pressure within a fluid bottle which either insufflates the working lumen or wash the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day. This may introduce multiple opportunities for contamination to the tube set by either contacting non-sterile surfaces or dropping the tubing on the floor.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a container arranged and configured to couple to an endoscope for use in an endoscopic procedure may comprise a container configured to contain a fluid within a first receptacle thereof, the container having a bottom portion and a top portion and movable between a collapsed storage configuration and an expanded use configuration, a water supply tube including a first end, a second end, and a first lumen extending therethrough, wherein the first lumen is in fluid communication with the first receptacle in the bottom portion of the container and the second end of the water supply tube is positioned external to the container, a gas supply tube including a first end, a second end, and a second lumen extending therethrough, wherein the second lumen is in operative fluid communication with the first receptacle and the second end of the gas supply tube is positioned external to the container, and a port positioned adjacent to the top portion of the container, wherein the port is configured to be in selective fluid communication with the first receptacle of the container.

Alternatively or additionally to any of the examples above, in another example, the container may further comprise a carrying handle adjacent the top portion of the container.

Alternatively or additionally to any of the examples above, in another example, the container may further comprise a second receptacle, the second receptacle may be configured to store the water supply tube and the gas supply tube when the container is the collapsed storage configuration.

Alternatively or additionally to any of the examples above, in another example, the second receptacle may be sealed prior to use of the container and unsealed at a time of use of the container.

Alternatively or additionally to any of the examples above, in another example, the port may comprise a removable cap.

Alternatively or additionally to any of the examples above, in another example, when in the expanded use configuration, the bottom portion of the container may be configured to support the container in an upright configuration.

Alternatively or additionally to any of the examples above, in another example, the container may further comprise comprising a support structure.

Alternatively or additionally to any of the examples above, in another example, in an absence of a biasing force, the support structure may be configured to maintain the container in the expanded use configuration.

Alternatively or additionally to any of the examples above, in another example, the support structure may comprise a support ring or pole.

Alternatively or additionally to any of the examples above, in another example, the support structure may comprise a planar reinforcing member movable from a first configuration to a second configuration.

Alternatively or additionally to any of the examples above, in another example, when the planar reinforcing member is in a first configuration the container may be in the collapsed storage configuration and when the planar reinforcing member is in the second configuration the container may be in the expanded use configuration.

Alternatively or additionally to any of the examples above, in another example, the support structure may extend from a first corner of the container adjacent the top portion thereof to a second corner of the container adjacent the bottom portion of the container.

Alternatively or additionally to any of the examples above, in another example, the first corner and the second corner may be at opposing lateral sides of the container.

Alternatively or additionally to any of the examples above, in another example, the container may further comprise a seal extending from the top portion of the container to the bottom portion of the container, the seal may be configured to divide the first receptacle into a first chamber and a second chamber.

Alternatively or additionally to any of the examples above, in another example, the container may further comprise an actuatable valve disposed between the first chamber and the second chamber.

Alternatively or additionally to any of the examples above, in another example, the container may further comprise a pressure relief valve in fluid communication with the second chamber.

Alternatively or additionally to any of the examples above, in another example, the port may be in fluid communication with the first chamber and the second chamber.

Alternatively or additionally to any of the examples above, in another example, the container may further comprise a cap configured to be releasably coupled to the port, the cap may be configured to selectively fluidly couple the first and second chambers.

Alternatively or additionally to any of the examples above, in another example, the seal may be selectively opened and closed, wherein when the seal is open the first and second chambers may be fluidly coupled and when the seal is closed the first and second chambers may be fluidly isolated from one another.

Alternatively or additionally to any of the examples above, in another example, the seal may comprise a fluid-tight hook and loop fastener.

In another example, a container arranged and configured to couple to an endoscope for use in an endoscopic procedure may comprise a flexible container configured to contain a fluid within a first receptacle thereof, the container having a bottom portion and a top portion and movable between a collapsed storage configuration and an expanded use configuration, a water supply tube including a first end, a second end, and a first lumen extending therethrough, wherein the first lumen is in fluid communication with the first receptacle in the bottom portion of the container and the second end of the water supply tube is positioned external to the container, a gas supply tube including a first end, a second end, and a second lumen extending therethrough, wherein the second lumen is in operative fluid communication with the first receptacle and the second end of the gas supply tube is positioned external to the container, a port positioned adjacent to the top portion of the container, wherein the port is configured to be in selective fluid communication with the first receptacle of the container, and a removable cap selectively coupled to the port.

Alternatively or additionally to any of the examples above, in another example, the container may further comprise a carrying handle adjacent the top portion of the container.

Alternatively or additionally to any of the examples above, in another example, the container may further comprise a second receptacle, the second receptacle may be configured to store the water supply tube and the gas supply tube when the container is the collapsed storage configuration.

Alternatively or additionally to any of the examples above, in another example, the second receptacle may be sealed prior to use of the container and unsealed at a time of use of the container.

Alternatively or additionally to any of the examples above, in another example, when in the expanded use configuration, the bottom portion of the container may be configured to support the container in an upright configuration.

In another example, a container arranged and configured to couple to an endoscope for use in an endoscopic procedure may comprise a container configured to contain a fluid within a first receptacle thereof, the container having a bottom portion and a top portion and movable between a collapsed storage configuration and an expanded use configuration, a water supply tube including a first end, a second end, and a first lumen extending therethrough, wherein the first lumen is in fluid communication with the first receptacle in the bottom portion of the container and the second end of the water supply tube is positioned external to the container, a gas supply tube including a first end, a second end, and a second lumen extending therethrough, wherein the second lumen is in operative fluid communication with the first receptacle and the second end of the gas supply tube is positioned external to the container, a port positioned adjacent to the top portion of the container, wherein the port is configured to be in selective fluid communication with the first receptacle of the container, and a support structure.

Alternatively or additionally to any of the examples above, in another example, in an absence of a biasing force, the support structure may be configured to maintain the container in the expanded use configuration.

Alternatively or additionally to any of the examples above, in another example, the support structure may comprise a support ring or pole.

Alternatively or additionally to any of the examples above, in another example, the support structure may comprise a planar reinforcing member movable from a first configuration to a second configuration.

Alternatively or additionally to any of the examples above, in another example, when the planar reinforcing member is in the second configuration, the support structure may be generally parallel to a bottom surface of the container.

Alternatively or additionally to any of the examples above, in another example, when the planar reinforcing member is in a first configuration the container may be in the collapsed storage configuration and when the planar reinforcing member is in the second configuration the container may be in the expanded use configuration.

Alternatively or additionally to any of the examples above, in another example, the support structure may extend from a first corner of the container adjacent the top portion thereof to a second corner of the container adjacent the bottom portion of the container.

Alternatively or additionally to any of the examples above, in another example, the first corner and the second corner may be at opposing lateral sides of the container.

In another example, a container arranged and configured to couple to an endoscope for use in an endoscopic procedure may comprise a container configured to contain a fluid therein, the container having a bottom portion and a top portion and movable between a collapsed storage configuration and an expanded use configuration, a water supply tube including a first end, a second end, and a first lumen extending therethrough, wherein the first lumen is in fluid communication with the bottom portion of the container and the second end of the water supply tube is positioned external to the container, a gas supply tube including a first end, a second end, and a second lumen extending therethrough, wherein the second lumen is in operative fluid communication with container and the second end of the gas supply tube is positioned external to the container, a port positioned adjacent to the top portion of the container, wherein the port is configured to be in selective fluid communication with an interior of the container, and a seal extending from the top portion of the container to the bottom portion of the container, the seal configured to divide the container into a first chamber and a second chamber.

Alternatively or additionally to any of the examples above, in another example, the container may further comprise an actuatable valve disposed between the first chamber and the second chamber.

Alternatively or additionally to any of the examples above, in another example, the container may further comprise a pressure relief valve in fluid communication with the second chamber.

Alternatively or additionally to any of the examples above, in another example, the port may be in fluid communication with the first chamber and the second chamber.

Alternatively or additionally to any of the examples above, in another example, the container may further comprise a cap configured to be releasably coupled to the port, the cap may be configured to selectively fluidly couple the first and second chambers.

Alternatively or additionally to any of the examples above, in another example, the seal may be selectively opened and closed, wherein the when the seal is open the first and second chambers are fluidly coupled and when the seal is closed the first and second chambers are fluidly isolated from one another.

Alternatively or additionally to any of the examples above, in another example, the seal may comprise a fluid-tight hook and loop fastener.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3A depicts an endoscope system with endoscope, light source, water reservoir, and tubing assembly for hybrid air, lens wash and irrigation fluid delivery, wherein the system is activated to deliver air to atmosphere;

FIG. 3B depicts the endoscope system of FIG. 3A, wherein the system is activated to deliver air to a patient through the patient end of the endoscope;

FIG. 3C depicts the endoscope system of FIG. 3A, wherein the system is activated to deliver lens wash fluid through the patient end of the endoscope;

FIG. 3D depicts the endoscope system of FIG. 3A, wherein the system is activated to deliver irrigation fluid through the patient end of the endoscope;

FIG. 4 depicts a hybrid endoscope system including a video processing unit, connector portion, peristaltic irrigation pump, water reservoir and top, coaxial gas and lens wash supply tubing, upstream and downstream irrigation supply tubing, and alternative gas supply tubing;

FIG. 5 depicts a perspective view of an illustrative refillable fluid reservoir;

FIG. 6A depicts a side view of an illustrative refillable fluid reservoir in a storage configuration;

FIG. 6B depicts the refillable fluid reservoir of FIG. 6A in a use configuration;

FIG. 7 depicts a perspective view of another illustrative refillable fluid reservoir;

FIG. 8 depicts a perspective view of another illustrative refillable fluid reservoir;

FIG. 9A depicts a side view of another illustrative refillable fluid reservoir in a first configuration;

FIG. 9B depicts the refillable fluid reservoir of FIG. 9A in a second configuration;

FIG. 10 depicts a schematic cross-sectional view of an illustrative cap for use with a refillable fluid reservoir;

FIG. 11A depicts a side view of another illustrative refillable fluid reservoir in a storage configuration; and

FIG. 11B depicts the refillable fluid reservoir of FIG. 11A in a use configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these capabilities compressed gasses from either the processor or alternative source are used to increase the pressure within a fluid bottle which either insufflates the working lumen or wash the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day. This may introduce multiple opportunities for contamination to the tube set by either contacting non-sterile surfaces or dropping the tubing on the floor. Disclosed herein are methods and systems to reduce or eliminate the need to disconnect the tube set and use a second bottle.

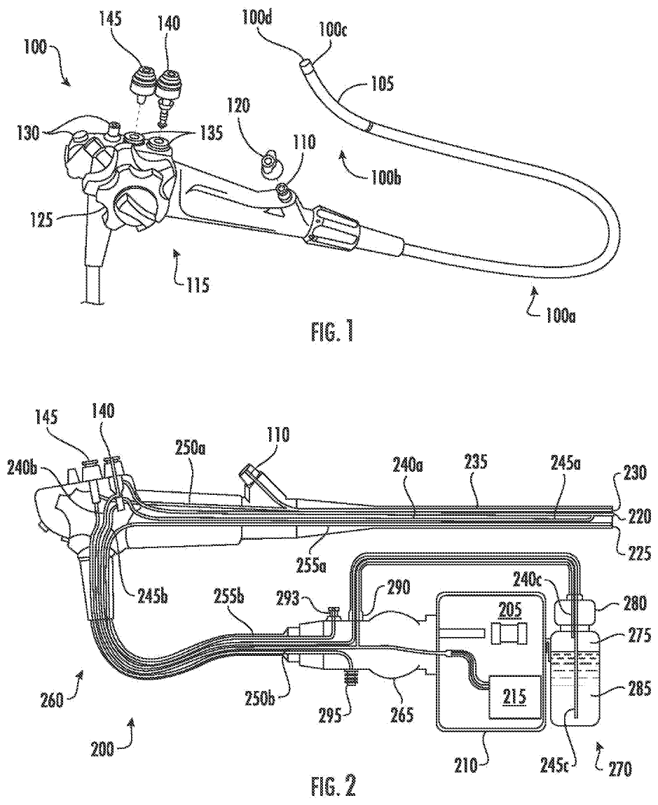

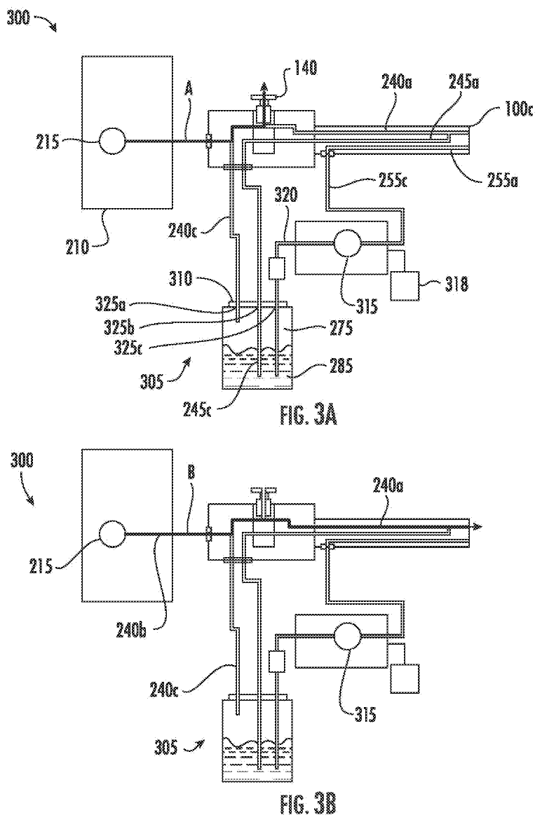

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connection portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

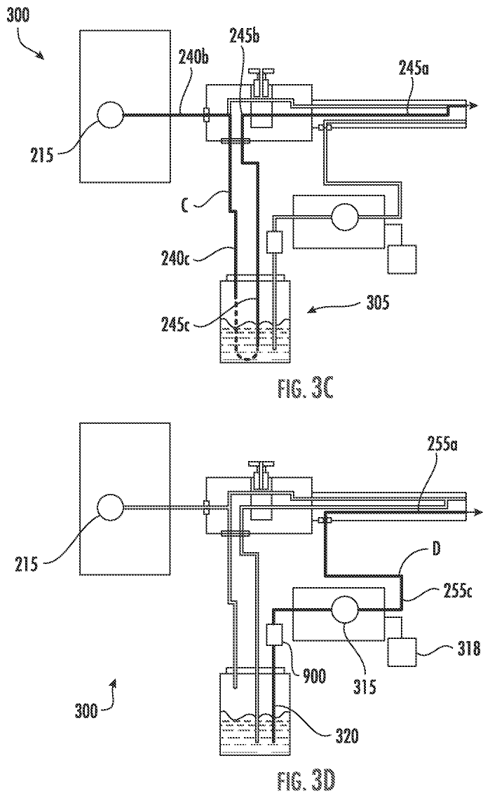

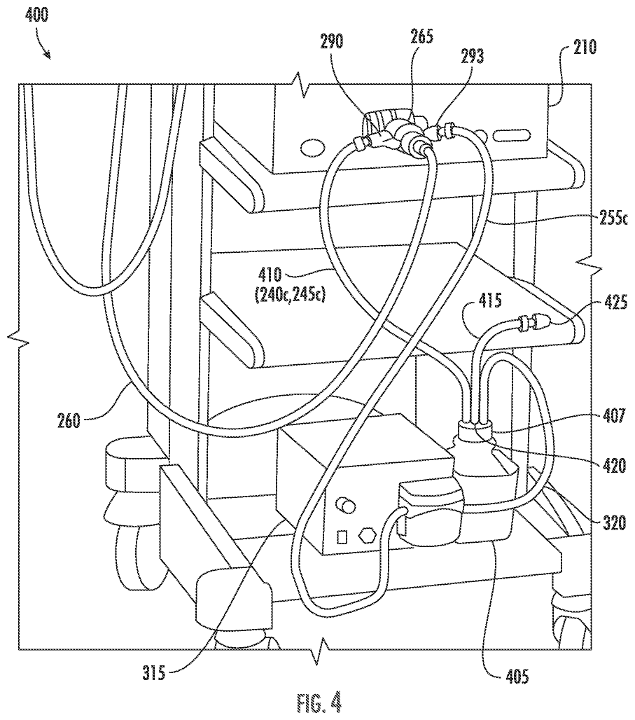

FIGS. 3A-3D are schematic drawings illustrating the operation of an embodiment of a hybrid system 300 where the supply tubing for irrigation and lens wash are connected to and drawn from a single water reservoir. It is contemplated that fluids other than water may be used, such as, but not limited to saline. The hybrid system 300 includes the single water reservoir 305, a cap 310 for the reservoir, gas supply tubing 240c, lens wash supply tubing 245c, irrigation pump 315 with foot switch 318, upstream irrigation tubing 320 and downstream irrigation supply tubing 255c. The cap 310 may be configured to attach in a seal-tight manner to the water reservoir 305 by a typically threaded arrangement. The cap 310 may include a gasket to seal the cap 310 to the reservoir 305. The gasket can be an O-ring, flange, collar, and/or the like and can be formed of any suitable material. A number of through-openings (325a, 325b, 325c) in the cap 310 are provided to receive, respectively, the gas supply tubing 240c, lens wash supply tubing 245c, and upstream irrigation supply tubing 320. In FIGS. 3A-3D, the system depicted includes separate tubing for gas supply, lens wash, and irrigation.

In other embodiments, the gas supply tubing 240c and lens wash tubing 245c may be combined in a coaxial arrangement. Some illustrative coaxial arrangements are described in commonly assigned U.S. patent application Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir (see, e.g., gas and lens wash supply tubing 240c, 245c). The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion (e.g., connector portion 265 of FIG. 2).

In various embodiments, different configurations of valving (not shown) may be incorporated into various embodiments disclosed hereby, including the tubing of the system 200, 300. For example, an in-flow check valve can be disposed in the path of the gas supply tubing 240c to help prevent backflow into the air pump 215. In this manner, pressure building within the water reservoir 305 creates a pressure difference between the water source and the gas supply tubing 240c helping to maintain a positive pressure in the water source even when large amounts of water may be removed from the water source during the irrigation function. This arrangement compensates for any time lag in air being delivered from the air pump 215 to the water reservoir 305, which might otherwise cause a negative pressure vacuum in the water reservoir. Similarly, an out-flow check valve, such as the one-way valve with inlet/outlets and valve insert, may be incorporated in the lens wash supply tubing 240c, upstream irrigation supply tubing 320, and/or downstream irrigation supply tubing 255c to help prevent backflow of water from either or both of the lens wash and irrigation tubing in the event of a negative pressure situation, as described.

More generally, in many embodiments, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check. Accordingly, a check valve as used herein is meant to be separate and distinct from an active valve that is operated in a binary manner as an on/off valve or switch to allowed flow to be turned on or allow flow to be turned off (e.g., a stop cock valve, solenoid valve, peristaltic pump).

During operation of the system of FIGS. 3A-3D, a flow of water for irrigation may be achieved by operating the irrigation pump 315. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 305, the pressure in the system may be controlled to maintain the lens wash supply tubing 240c at substantially the pressure necessary to accomplish a lower flow rate lens wash, while compensating for reduced pressure in the water reservoir 305 due to supplying a high flow rate irrigation. When pressure is reduced in the water reservoir by use of the lens wash function, the irrigation function, or both functions simultaneously, the reduced pressure may be compensated for by the air pump 215 via the gas supply tubing 240c.

The schematic set-up in FIGS. 3A-3D has been highlighted to show the different flow paths possible with the hybrid system 300 having supply tubing for irrigation 320 and lens wash 240c connected to and drawn from the single water reservoir 305. As shown in FIG. 3A, the endoscope 100 is in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b in the umbilical 260 via the connector portion 265 and through the gas/water valve to atmosphere. Since the system is open at the vent hole in the gas/water valve 140, there is no build up to pressurize the water reservoir 305 and consequently no water is pushed through the lens wash supply tubing 240c.

As shown in FIG. 3B, the endoscope 100 is in a gas delivery state with the gas/water valve 140 in a first position. When gas is called for at the distal tip 100c, for example, to clean the end face 100d of the distal tip or insufflate the patient body in the treatment area, the user closes off the vent hole in the gas/water valve 140 with a thumb, finger, or the like (first position). In this state, gas (pressure) is delivered along path B from the air pump 215 and flowed through the gas feed line 240b in the umbilical 260 via the connector portion 265. The gas continues through the gas/water valve 140 to the gas supply line 240a in the endoscope shaft 100a and out the gas/lens wash nozzle 220 at the distal tip 100c. There is no build up to pressurize the water reservoir since the system is open at the gas/lens water nozzle 220, and consequently no water is pushed through the lens wash supply tubing 240c.

As shown in FIG. 3C, the endoscope 100 is in a lens wash delivery state with the gas/water valve 140 in a second position. When lens wash is called for at the distal tip 100c, for example, to clean the end face 100d of the distal tip 100c, the user, keeping the vent hole in the air/water valve closed off, depresses the valve 140 to its furthest point in the valve well 135. The second position blocks off the gas supply to both atmosphere and the gas supply line 240a in the endoscope, and opens up the gas/water valve 140 to allow lens wash water to pass through to the lens wash supply line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 305. The gas (pressure) pressurizes the surface of the remaining water 285 in the reservoir 305 and pushes water up the lens wash supply tube 245c to the connector portion 265. The pressurized lens wash water is pushed further through the lens wash feed line 245b in the umbilical 260 and through the gas/water valve 140. Since the system 300 is closed, gas pressure is allowed to build and maintain a calibrated pressure level in the water reservoir 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user activates the irrigation pump 315 (e.g., by depressing foot switch 318) to delivery water along path D. With the pump 315 activated, water is sucked out of the water reservoir 305 through the upstream irrigation supply tubing 320 and pumped along the downstream irrigation supply tubing 255c to the connector portion 265. The irrigation pump head pressure pushes the irrigation water further through the irrigation feed line 255b in the umbilical 260, through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

FIG. 4 is a schematic drawing illustrating a further embodiment of a hybrid system 400 including a video processing unit 210, connector portion 265, peristaltic irrigation pump 315, water reservoir 405 and top 407, coaxial gas and lens wash supply tubing 410, upstream and downstream irrigation supply tubing 320, 255c, and alternative gas supply tubing 415 (e.g., CO₂). A length of the alternative gas supply tubing 415 passes from one end positioned in the gas gap 275 between the top 407 of the water reservoir 405 and the remaining water 285 in the reservoir through an additional opening 420 in the top of the reservoir to a detachable connection 425 for a source of the alternative gas supply (e.g., CO₂ hospital house gas source). When the alternative gas supply is desired, such as CO₂ gas, the air pump 215 on the video processing unit 210 may be turned off and CO₂ gas, rather than air, is thereby flowed to the water reservoir 405 pressurizing the water surface. In the neutral state, CO₂ gas flows backward up the gas supply tubing 240c to the connector portion 265, up the gas feed line 240b, and is vented through the gas/water valve 140 to atmosphere. In the first position, the user closes off the vent hole in the gas/water valve 140, and the CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the reservoir 405 is maintained by delivery gas through alternative gas (e.g., CO₂) supply tubing 415. The irrigation function may be accomplished in a similar manner as the operation described above with respect to FIG. 3D.

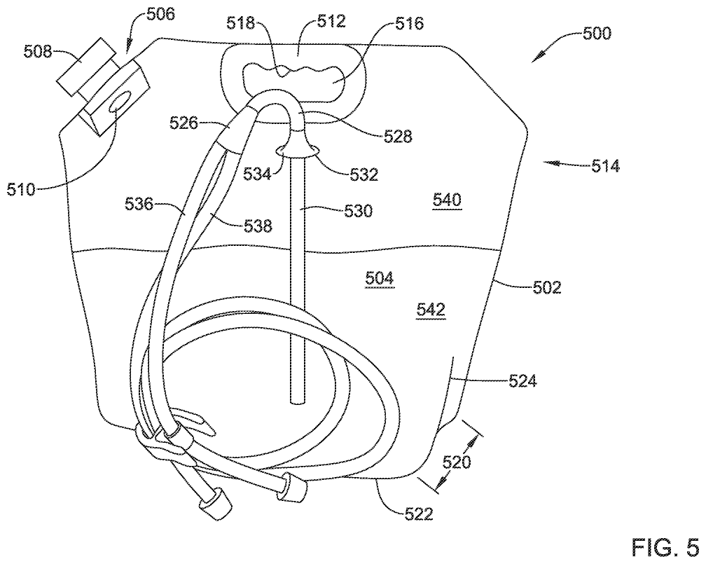

As described above, it may be desirable to reduce opportunities for contamination to the tube set 240c, 245c, 320, 410, 415 during replacement of the water reservoir by providing a refillable water reservoir 270, 305, 405. FIG. 5 depicts a perspective view of an illustrative refillable fluid reservoir 500. The reservoir 500 may be configured to be used in an endoscopic system and includes components similar to the endoscope and endoscope systems described with regard to FIGS. 1-4; however, not all features may be described or shown here if not pertinent to the fluid circuit of the system. The reservoir 500 includes a container 502 defining a first receptacle 504 configured to hold a fluid 542. The container 502 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In some embodiments, the container 502 may be entirely translucent, entirely opaque, or combinations thereof. The reservoir 500 may further include a port 506 having a removable cap 508. The cap 508 may be formed from more rigid material (relative to the container 502) and may be configured to form a fluid tight seal with the port 506. The cap 508 may be configured to threadably engage the port 506, form a friction fit with the port 506, form a snap fit with the port 506 or otherwise releasably engage the port 506. In some examples, the port 506 and/or cap 508 may be formed from polyethylene terephthalate (PET), polypropylene (PP), etc. Portions of the port 506 may extend into the first receptacle 504. The removable cap 508 may be removed to place a fluid source in selective fluid communication with the first receptacle 504 and allow fluid to be poured through a lumen 510 of the port 506 and into the first receptacle 504.

The reservoir 500 may include a carrying handle 512 positioned adjacent to a top portion 514 thereof. The handle 512 may define an opening or through hole 516 for receiving a hand or hook therethrough to carry the reservoir 500. In some cases, the carrying handle 512 may include an undulating carrying surface 518 configured to provide a more ergonomic grip for the user. It is contemplated that the handle 512 may be formed from a similar material as the cap 508 or the container 502, as desired. In some examples, the handle 512 may be formed from polyethylene terephthalate (PET), polypropylene (PP), etc.

The reservoir 500 may be movable between a collapsed storage configuration (not explicitly shown) and an expanded use configuration (FIG. 5). In the expanded use configuration, the reservoir 500 may increase in width 520 from the top portion 514 towards the bottom portion 522. In the use configuration, the bottom portion 522 may have a width that allows the reservoir 500 to remain upright without user intervention. The bottom portion 522 may include folds or pleats 524 that allow the bottom portion 522 to fold or collapse. In the collapsed storage configuration, the top portion 514 and the bottom portion 522 may have a similar width 520 which allows the reservoir 500 to lay substantially flat such that the reservoir 500 may be stacked with other fluid reservoirs 500. In other examples, the reservoir 500 may be rolled or folded to reduce the amount of storage space it occupies. In some cases, since the reservoir 500 is sealed or capable of being sealed, a vacuum may be pulled during packaging of the reservoir 500 to further reduce the storage space required to store the reservoir 500. In some embodiments, the reservoir 500 may be stored inside out such that the tubing 536, 538, manifold 526, fittings and/or other components are within the first receptacle 504. When the reservoir 500 is taken from storage for use, the entire inside of the reservoir 500 may be pulled through the port 506 and the tubes 536, 538 connected to the connector portion 265 of the umbilical 260. The reservoir 500 may be stored in sterile packaging to maintain sterility of the tubing 536, 538 and interior surface of the first receptacle 504.

The reservoir 500 may be connected in fluid communication with a tubing manifold 526 via a shared length of gas supply/alternate gas supply tubing (or gas supply tubing) 528 and lens wash supply/irrigation supply tubing 530 (or water supply tubing 530). The shared gas supply tubing 528 extends from a second end at an opening of the manifold 526 (external to the reservoir 500) through a reservoir opening 532 in the top portion 514 of the first receptacle 504. The shared gas supply tubing 528 may terminate within a reservoir interior 540, at or below the opening 532, but not extending into the remaining fluid 542 in the first receptacle 504. However, in some cases, the gas supply tubing 528 may extend into or be adjacent to the fluid 542. For example, the opening 532 may be at a bottom portion 522 or side of the container 502 such that the shared gas supply tubing 528 terminates within the fluid with gas bubbling up through the fluid 542 to pressurize the container 502. A lumen extends through the gas supply tubing 528 for receiving a flow of air and/or gas therethrough. The lumen of the gas supply tubing 528 is in operative fluid communication with the interior of the reservoir 500. The water supply tubing 530 extends from a second end at an opening of manifold 526 (external to the reservoir 500) through the reservoir opening 532, terminating in a first end within the remaining fluid 542 at or substantially at the bottom of the container 502. A lumen extends through the water supply tubing 530 for receiving a flow of fluid therethrough. In some embodiments, the water supply tubing 530 may terminate at the opening 532. For example, when the opening 532 is at or adjacent to the bottom portion 522 of the container 502 a dip tube may not be required. The lumen of the lens wash supply/irrigation supply tubing 530 is in selective operative fluid communication with the bottom portion of the container 502. In the illustrated embodiment, the gas supply tubing 528 and the water supply tubing 530 may enter the container 502 through a single or common opening 532. For example, the gas supply tubing 528 and the water supply tubing 530 may be coaxially arranged. However, this is not required. In some cases, the gas supply tubing 528 and the water supply tubing 530 may extend in a side by side arrangement or may be separately connected to the container 502 in different locations. The opening 532 may include a grommet or heat seal 534 configured to seal the container 502 about the tubing 528, 530 in a fluid and pressure tight manner.

A portion of a gas supply tubing 536 and a portion of a lens wash supply tubing 538 may extend from the manifold 526, and may be connected in fluid communication with the endoscope at gas/lens wash connection on the connector portion 265 of the umbilical. The portion of the gas supply tubing 536 is connected in fluid communication with a gas pump (not explicitly shown) and gas feed line (not explicitly shown), and the portion of the lens wash supply tubing 538 is connected in fluid communication with lens wash feed line (not explicitly shown), within connector portion 265. The portion of the gas supply tubing 536 is in fluid communication through tubing manifold 526 with the shared gas supply tubing 528. Similarly, the portion of the lens wash supply tubing 538 is in fluid communication through tubing manifold 526 with the shared lens wash/irrigation (or water) supply tubing 530. While not explicitly shown, irrigation supply tubing may be coupled to the manifold to supply irrigation fluid from the reservoir 500.

It is contemplated that the reservoir 500 may be filled and refilled as needed by removing the cap 508 and pouring water into the first receptacle 504. The refilling of the reservoir 500 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 500 with sterile or non-sterile water may create more flexibility and reduce the need to have as much sterile water in storage. Further, refilling the reservoir 500 via the port 506 and removable cap 508 may also remove the need to disconnect the reservoir 500 from the tubing 528, 530 throughout the day eliminating or greatly reducing the possibility of cross contamination by removing the need to replace the water container.

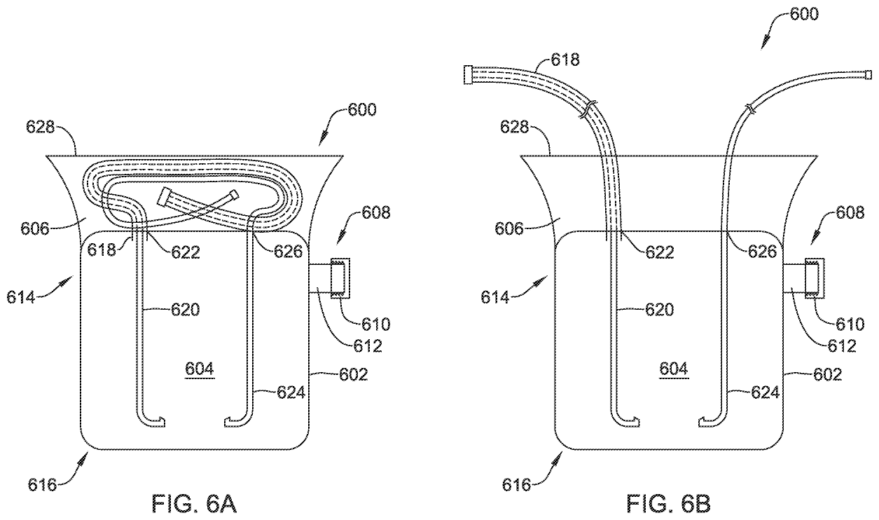

FIG. 6A depicts a side view of an illustrative refillable fluid reservoir 600 in a storage configuration and FIG. 6B depicts the refillable fluid reservoir 600 in a use configuration. The reservoir 600 may be configured to be used in an endoscopic system and includes components similar to the endoscope and endoscope systems described with regard to FIGS. 1-4; however, not all features may be described or shown here if not pertinent to the fluid circuit of the system. The reservoir 600 includes a container 602 defining a first receptacle 604 and a second receptacle 606. The first and second receptacles 604, 606 may be sealed such that fluid does not pass directly between the first and second receptacles 604, 606. The container 602 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In some embodiments, the container 602 may be entirely translucent, entirely opaque, or combinations thereof. The reservoir 600 may further include a port 608 having a removable cap 610. The cap 610 may be formed from more rigid material (relative to the container 602) and may be configured to form a fluid tight seal with the port 608. The cap 610 may be configured to threadably engage the port 608, form a friction fit with the port 608, form a snap fit with the port 608 or otherwise releasably engage the port 608. In some examples, the port 608 and/or cap 610 may be formed from polyethylene terephthalate (PET), polypropylene (PP), etc. Portions of the port 608 may extend into the first receptacle 604. The removable cap 610 may be removed to place a fluid source in selective fluid communication with the first receptacle 604 and allow fluid to be poured through a lumen 612 of the port 608 and into the first receptacle 604.

While not explicitly shown, the reservoir 600 may include a handle or other carrying feature positioned adjacent to the second receptacle 606 thereof. The handle may define an opening or through hole for receiving a hand therethrough to carry the reservoir 600. In some cases, the carrying handle may include an ergonomic grip for the user. It is contemplated that the handle may be formed from a similar material as the cap 610 or may be formed from the same material as the container 602. In some examples, the handle may be formed from polyethylene terephthalate (PET), polypropylene (PP), etc.

The reservoir 600 may be connected in fluid communication with a lumen of a gas supply tube 618 and a lumen of a water supply tube 620. The gas supply tube 618 and the water supply tube 620 may be provided in a shared length of tubing. The gas supply tube 618 and the water supply tube 620 may be coaxially arranged with the water supply tube 620 extending within and through the lumen of the gas supply tube 618 along a portion of the length of the gas supply tube 618. However, this is not required. In some cases, the gas supply tube 618 and the water supply tube 620 may extend side by side. The gas supply tubing 618 extends from a second end to a first end adjacent to an opening 622 in the first receptacle 604. In the use configuration, the second end of the gas supply tubing 618 may be external to the reservoir 600 while in the storage configuration, the second end of the gas supply tubing 618 may be stored in the second receptacle 606. A lumen extends through the gas supply tube 618 for receiving a flow of air and/or gas therethrough. The lumen of the gas supply tube 618 is in fluid communication with the first receptacle 604. The first end of the gas supply tubing 618 is in selective fluid communication with the top portion 614 of the first receptacle 604 in the embodiment shown. In other embodiments, the gas supply tubing 618 may be connected at other regions of the first receptacle 604, such as, but not limited to, a bottom portion 616 or a side portion. The water supply tubing 620 extends from a second end to a first end which extends through the opening 622 and into an interior of the first receptacle 604. In the use configuration, the second end of the water supply tubing 620 may be external to the reservoir 600 while in the storage configuration, the second end of the water supply tubing 620 may be stored in the second receptacle 606. A lumen extends through the water supply tube 620 for receiving a flow of fluid therethrough. The second ends of the gas supply tube 618 and the water supply tube 620 may be coupled to a manifold (if so provided) or a connector portion 265 of an endoscope system. The first end of the water supply tube 620 is in selective fluid communication with the bottom portion 616 of the first receptacle 604. The opening 622 may include a grommet or heat seal configured to seal the container 602 about the tubing 618, 620 in a fluid and pressure tight manner.

The opening 622 is positioned in the top portion 614 of the first receptacle 604 in the embodiment shown. However, in other embodiments, the opening 622 is may be positioned in the bottom portion 616 of the first receptacle 604, thereby obviating the need to extend a substantial length of the water supply tube 620 into the first receptacle 604. In these embodiments, the gas supply tubing 618 will be in selective fluid communication with the bottom portion 616 of the first receptacle 604 with gas bubbling to the air space above during use.

In some embodiments, the reservoir 600 may be connected in fluid communication with a lumen of an irrigation supply tube 624. The irrigation supply tubing 624 extends from a second end to a first end which extends through an opening 626 and into an interior of the first receptacle 604. In the use configuration, the second end of the irrigation supply tubing 624 may be external to the reservoir 600 while in the storage configuration, the second end of the irrigation supply tubing 624 may be stored in the second receptacle 606. A lumen extends through the irrigation supply tube 624 for receiving a flow of fluid therethrough. In some cases, the irrigation supply tube 624 may be coupled to a manifold, if so provided. The first end of the irrigation supply tube 624 is in selective fluid communication with the bottom portion 616 of the first receptacle 604. The opening 626 may include a grommet or heat seal configured to seal the container 602 about the tubing 624 in a fluid and pressure tight manner. The opening 626 is positioned in the top portion 614 of the first receptacle 604 in the embodiment shown. However, in other embodiments, the opening 626 is may be positioned in the bottom portion 616 of the first receptacle 604, thereby obviating the need to extend a substantial length of the irrigation supply tube 624 into the first receptacle 604.

The second ends of the gas supply tubing 618 and the lens wash supply tubing 620 may be connected in fluid communication with the endoscope at gas/lens wash connection on the connector portion 265 of the umbilical. The gas supply tubing 618 is connected in fluid communication with a gas pump (not explicitly shown) and gas feed line (not explicitly shown), and the lens wash supply tubing 620 is connected in fluid communication with lens wash feed line (not explicitly shown), within connector portion 265. The irrigation tubing 624 is connected in fluid communication with the irrigation supply line 255c via an irrigation pump 315.

The reservoir 600 may be movable between a collapsed storage configuration and an expanded use configuration. In the storage configuration, the gas supply tube 618, water supply tube 620, and the irrigation supply tube 624 may be stored within the second receptacle 606. For example, the tubes 618, 620, 624 may be placed in the second receptacle 606 after sterilization of the tubes 618, 620, 624 and at least the interior of first and second receptacles 604, 606. After the tubes 618, 620, 624 have been placed in the second receptacle 606, the top 628 of the second receptacle 606 may be sealed to maintain the tubes 618, 620, 624 in a sterile environment. In some examples, the top 628 of the second receptacle 606 may include a seal that is configured to be resealed such as, but not limited to, a zip top seal, hook and loop closures, etc. Providing a resealable second receptacle 606 may allow the tubes 618, 620, 624 to be stored between uses or conveniently stowed away for disposal. Additionally, or alternatively, the top 628 of the second receptacle 606 may include perforations to allow the second receptacle 606 to be torn open. It is contemplated that storing the tubes 618, 620, 624 in the second receptacle 606 during shipping and/or storage may eliminate the need for the reservoir 600 to be disposed within a sterile shipping pouch as the exterior of the reservoir 600 does not need to be sterile.

While the second receptacle 606 is illustrated as being positioned adjacent the top portion 614 of the container 602, the second receptacle 606 may be positioned along a lateral side or adjacent the bottom portion 616 of the container, if so desired. For example, the second receptacle 606 may be positioned adjacent the bottom portion 616 of the container in embodiments where the opening 622 and/or the opening 626 is/are positioned in the bottom portion 616 of the first receptacle 604. In the collapsed storage configuration, the top portion 614 and the bottom portion 616 may have a similar width which allows the reservoir 600 to lay substantially flat such that the reservoir 600 may be stacked with other fluid reservoirs 600. In other examples, the reservoir 600 may be rolled or folded to reduce the amount of storage space it occupies. In some cases, since the reservoir 600 is sealed or capable of being sealed, a vacuum may be pulled during packaging of the reservoir 600 to further reduce the storage space required to store the reservoir 600.

To use the reservoir 600, the second receptacle 606 may be opened or unsealed and the tubing 618, 620, 624 removed and coupled to the endoscope system. The first receptacle 604 may be filled with sterile or non-sterile water via the port 608. In the expanded use configuration, the reservoir 600 may increase in width from the top portion 614 towards the bottom portion 616 similar to the reservoir 500 described herein. In the use configuration, the bottom portion 616 may have a width that allows the reservoir 600 to remain upright without user intervention. The bottom portion 616 may include folds or pleats that allow the bottom portion 616 to fold or collapse.

It is contemplated that the reservoir 600 may be filled and refilled as needed by removing the cap 610 and pouring water into the first receptacle 604. The refilling of the reservoir 600 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 600 with sterile or non-sterile water may create more flexibility and reduce the need to have as much sterile water in storage. Further, refilling the reservoir 600 via the port 608 and removable cap 610 may also remove the need to disconnect the reservoir 600 from the tubing 618, 620, 624 throughout the day eliminating or greatly reducing the possibility of cross contamination by removing the need to replace the water container.

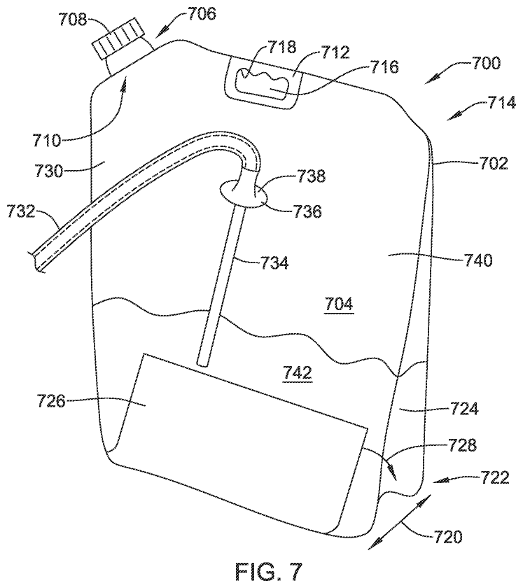

FIG. 7 depicts a perspective view of an illustrative refillable fluid reservoir 700. The reservoir 700 may be configured to be used in an endoscopic system and includes components similar to the endoscope and endoscope systems described with regard to FIGS. 1-4; however, not all features may be described or shown here if not pertinent to the fluid circuit of the system. The reservoir 700 includes a container 702 defining a first receptacle 704 configured to hold a fluid 742. The container 702 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In some embodiments, the container 702 may be entirely translucent, entirely opaque, or combinations thereof. The reservoir 700 may further include a port 706 having a removable cap 708. The cap 708 may be formed from more rigid material (relative to the container 702) and may be configured to form a fluid tight seal with the port 706. The cap 708 may be configured to threadably engage the port 706, form a friction fit with the port 706, form a snap fit with the port 706 or otherwise releasably engage the port 706. In some examples, the port 706 and/or cap 708 may be formed from polyethylene terephthalate (PET), polypropylene (PP), etc. Portions of the port 706 may extend into the first receptacle 704. The removable cap 708 may be removed to a fluid source in selective fluid communication with the first receptacle 704 and allow fluid to be poured through a lumen 710 of the port 706 and into the first receptacle 704.

The reservoir 700 may include a carrying handle 712 or other carrying mechanism positioned adjacent to a top portion 714 thereof. The handle 712 may define an opening or through hole 716 for receiving a hand or hook therethrough to carry the reservoir 700. In some cases, the carrying handle 712 may include an undulating carrying surface 718 configured to provide a more ergonomic grip for the user. It is contemplated that the handle 712 may be formed from a similar material as the cap 708 or from a same material as the container 702. In some examples, the handle 712 may be formed from polyethylene terephthalate (PET), polypropylene (PP), etc.

The reservoir 700 may be movable between a collapsed storage configuration (not explicitly shown) and an expanded use configuration (FIG. 7). In the expanded use configuration, the reservoir 700 may increase in width 720 from the top portion 714 towards the bottom portion 722. In the use configuration, the bottom portion 722 may have a width that allows the reservoir 700 to remain upright without user intervention. The bottom portion 722 may include folds or pleats 724 that allow the bottom portion 722 to fold or collapse. The bottom portion 722 may further include a planar reinforcing member or structural stiffener 726. The stiffener 726 may be movable from a first plane or configuration in a collapsed configuration to a second plane or configuration in the expanded configuration, as shown at arrow 728. In some embodiments, the second plane may be generally perpendicular to the first plane. The stiffener 726 may be formed from a material that is more rigid than the material of the container 702. In the expanded configuration, the stiffener 726 may add structure to the bottom surface of the container 702 which was collapsed or folded in the storage configuration.

In the collapsed storage configuration, the top portion 714 and the bottom portion 722 may have a similar width 720 which allows the reservoir 700 to lay substantially flat such that the reservoir 700 may be stacked with other fluid reservoirs 700. In this configuration, the stiffener 726 may lie generally parallel to a front surface 730 of the reservoir 700. In other examples, the reservoir 700 may be folded to reduce the amount of storage space it occupies. In some cases, since the reservoir 700 is sealed or capable of being sealed, a vacuum may be pulled during packaging of the reservoir 700 to further reduce the storage space required to store the reservoir 700. In some embodiments, the reservoir 700 may be stored inside out such that the tubing 732, 734, fittings and/or other components are within the first receptacle 704. When the reservoir 700 is taken from storage for use, the entire inside of the reservoir 700 may be pulled through the port 706 and the tubes 732, 734 connected to the connector portion 265 of the umbilical 260. The reservoir 700 may be stored in sterile packaging to maintain sterility of the tubing 732, 734 and interior surface of the first receptacle 704. In some embodiments, the tubing 732, 734 may be stored within a second receptacle (not shown) as previously described.

The reservoir 700 may be connected in fluid communication with a gas supply/alternate gas supply tubing (or gas supply tubing) 732 and a lens wash supply/irrigation supply tubing (or water supply tubing) 734. The gas supply tubing 732 extends from a second end external to the reservoir 700 through a reservoir opening 736 in the top portion 714 of the first receptacle 704. The shared gas supply tubing 732 may terminate within a reservoir gap 740, at or below the opening 736, but not extending into the remaining fluid 742 in the first receptacle 704. However, in some cases, the gas supply tubing 732 may extend into the fluid 742. For example, the opening 736 may be at a bottom or side of the container 702 such that the shared gas supply tubing 732 terminates within the fluid with gas bubbling up through the fluid 742 to pressurize the container 702. A lumen extends through the gas supply tubing 732 for receiving a flow of air and/or gas therethrough. The lumen of the gas supply tubing 732 is in operative fluid communication with an interior of the reservoir 700. The water supply tubing 734 extends from a second end external to the reservoir 700 through the reservoir opening 736, terminating in a first end within the remaining fluid 742 at or substantially at the bottom of the container 702. In some embodiments, the water supply tubing 734 may terminate at the opening 736. For example, when the opening 736 is at or adjacent to the bottom portion 722 of the container 702 a dip tube may not be required. A lumen extends through the water supply tubing 734 for receiving a flow of fluid therethrough. The lumen of the lens wash supply/irrigation supply tubing 734 is in selective operative fluid communication with the bottom portion of the container 702. In the illustrated embodiment, the gas supply tubing 732 and the water supply tubing 734 may enter the container 702 through a single or common opening 736. For example, the gas supply tubing 732 and the water supply tubing 734 may be coaxially arranged. However, this is not required. In some cases, the gas supply tubing 732 and the water supply tubing 734 may extend in a side by side arrangement or may be separately connected to the container 702 in different locations. The opening 736 may include a grommet or heat seal 738 configured to seal the container 702 about the tubing 732, 734 in a fluid and pressure tight manner.

A portion of a gas supply tubing 732 and a portion of lens wash supply tubing 734 may extend from the reservoir 700, respectively, and may be connected in fluid communication with the endoscope at gas/lens wash connection on the connector portion 265 of the umbilical. The gas supply tubing 732 is connected in fluid communication with a gas pump (not explicitly shown) and gas feed line (not explicitly shown), and the lens wash supply tubing 734 is connected in fluid communication with lens wash feed line (not explicitly shown), within connector portion 265. While not explicitly shown, irrigation supply tubing may be coupled to the water supply tubing 734 via a manifold to supply irrigation fluid from the reservoir 700 or a separate irrigation supply tubing may be provided.

It is contemplated that the reservoir 700 may be filled and refilled as needed by removing the cap 708 and pouring water into the first receptacle 704. The refilling of the reservoir 700 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 700 with sterile or non-sterile water may create more flexibility and reduce the need to have as much sterile water in storage. Further, refilling the reservoir 700 via the port 706 and removable cap 708 may also remove the need to disconnect the reservoir 700 from the tubing 732, 734 throughout the day eliminating or greatly reducing the possibility of cross contamination by removing the need to replace the water container.

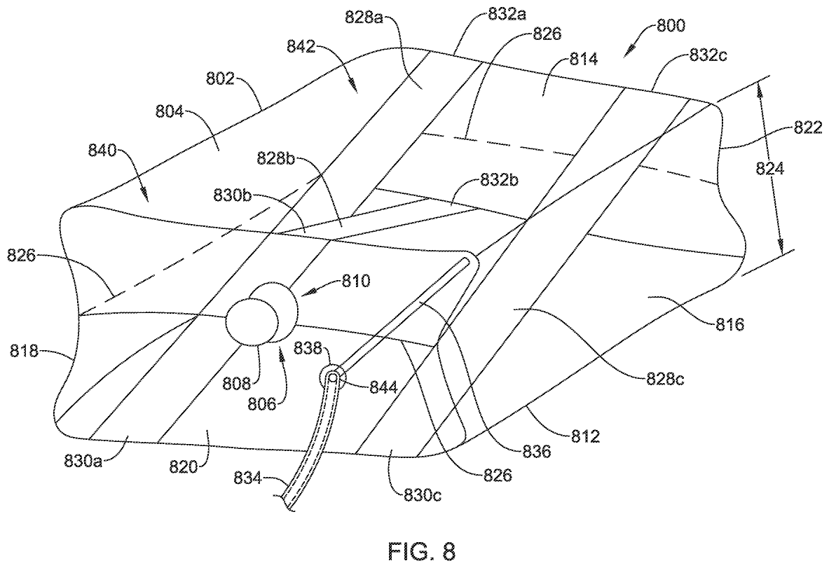

FIG. 8 depicts a perspective view of an illustrative refillable fluid reservoir 800 resting on a back side 812. The reservoir 800 may be configured to be used in an endoscopic system and includes components similar to the endoscope and endoscope systems described with regard to FIGS. 1-4; however, not all features may be described or shown here if not pertinent to the fluid circuit of the system. The reservoir 800 includes a container 802 defining a first receptacle 804 configured to hold a fluid. The container 802 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In some embodiments, the container 802 may be entirely translucent, entirely opaque, or combinations thereof. The reservoir 800 may further include a port 806 having a removable cap 808. The port 806 may be positioned adjacent a top portion 840 of the container 802. The cap 808 may be formed from more rigid material (relative to the container 802) and may be configured to form a fluid tight seal with the port 806. The cap 808 may be configured to threadably engage the port 806, form a friction fit with the port 806, form a snap fit with the port 806 or otherwise releasably engage the port 806. In some examples, the port 806 and/or cap 808 may be formed from polyethylene terephthalate (PET), polypropylene (PP), etc. Portions of the port 806 may extend into the first receptacle 804. The removable cap 808 may be removed to place a fluid source in selective fluid communication with the first receptacle 804 and allow fluid to be poured through a lumen 810 of the port 806 and into the first receptacle 804.

The reservoir 800 may include a carrying handle or other carrying mechanism (not explicitly shown). The handle may define an opening or through hole for receiving a hand or hook therethrough to carry the reservoir 800. In some cases, the carrying handle may include an ergonomic grip for the user. It is contemplated that the handle may be formed from a similar material as the cap 808 or from a same material as the container 802. In some examples, the handle may be formed from polyethylene terephthalate (PET), polypropylene (PP), etc.

The illustrative reservoir 800 includes a front side 814, a back side 812, and at least a first side 816, and a second opposing side 818. The first and second sides 816, 818 may each extend from or between the front 814 to the back 812. The reservoir 800 may further include a top 820 and an opposing bottom 822. The top and bottom 820, 822 may extend from or between the first and second sides 816, 818. The use of the terms “front”, “back”, “first”, “second”, “top”, and “bottom” are not intended to limit the reservoir 800 to a particular orientation, but rather facilitate discussion of relative orientation. Further, the reservoir 800 is not limited to a rectangular or generally rectangular structure. Other shapes may be used for the reservoir 800, as desired.

The reservoir 800 may be movable between a collapsed storage configuration (not explicitly shown) and an expanded use configuration (FIG. 8). In the expanded use configuration, the reservoir 800 may have a width 824 that is approximately constant from the top 820 towards the bottom 822. In the use configuration, the bottom 822 may have a width that allows the reservoir 800 to remain upright without user intervention (e.g., with the port 806 and cap 808 upwards). The top 820, bottom 822, first side 816, and/or second side 818 may include folds or pleats 826 that allow the container 802 to fold or collapse. The container 802 may further include one or more support structures or structural stiffeners 828a, 828b, 828c (collectively, 828). While the container 802 is illustrated as including three stiffeners 828, the container 802 may include more than three or fewer than three stiffeners 828, as desired. In some examples, the stiffeners 828 may extend diagonally (e.g., at a non-orthogonal and non-parallel angle) through the first receptacle 804, although orthogonal and parallel extending stiffeners 828 may be used. Generally, the support structures 828 may extends from a first corner of the container 802 adjacent the top 820 thereof to a second corner of the container 802 adjacent the bottom 822 of the container 802. In some cases, the first and second corners may be at opposing lateral sides of the container 802 (e.g., front 814 and back 812). For example, one or more stiffeners 828a, 828c may have a first end 830a, 830c adjacent to a corner of the top 820 and the back 812 and a second end 832a, 832c adjacent to a corner of the bottom 822 and the front 814. One or more additional stiffeners 828b may have a first end 830b adjacent to a corner of the top 820 and the front 814 and a second end 832b adjacent to a corner of the bottom 822 and the back 812. The internal stiffeners 828 may be positioned in other configurations or orientations, as desired. It is contemplated that the internal stiffeners 828 may provide additional stability to the form of the reservoir 800 once the first receptacle 804 is filled with fluid. The additional stability may allow the reservoir to lie neatly on a shelf and/or allow multiple reservoirs 800 to be stacked one on top of another to minimize space required and handling.

In the collapsed storage configuration, the top 820 and the bottom 822 may have a similar width 824 which allows the reservoir 800 to lay substantially flat such that the reservoir 800 may be stacked with other fluid reservoirs 800. In this configuration, the stiffeners 828 may lie generally parallel to the front 814 and/or back 812 of the reservoir 800. In other examples, the reservoir 800 may be folded to reduce the amount of storage space it occupies. In some cases, since the reservoir 800 is sealed or capable of being sealed, a vacuum may be pulled during packaging of the reservoir 800 to further reduce the storage space required to store the reservoir 800. In some embodiments, the tubing 834, 836 may be stored within a second receptacle (not shown) as previously described.

The reservoir 800 may be connected in fluid communication with a gas supply/alternate gas supply tubing (or gas supply tubing) 834 and a lens wash supply/irrigation supply tubing 836. The gas supply tubing 834 extends from a second end external to the reservoir 800 through a reservoir opening 838 in the top 820 of the first receptacle 804. The shared gas supply tubing 834 may terminate within a reservoir gap, at or below the opening 838, but not extending into the remaining fluid in the first receptacle 804. However, in some cases, the gas supply tubing 834 may extend into the fluid. For example, the opening 838 may be at a bottom or side of the container 802 such that the shared gas supply tubing 834 terminates within the fluid with gas bubbling up through the fluid to pressurize the container. A lumen extends through the gas supply tubing 834 for receiving a flow of air and/or gas therethrough. The lumen of the gas supply tubing 834 is in operative fluid communication with a top portion 840 of the reservoir 800. The water supply tubing 836 extends from a second end external to the reservoir 800 through the reservoir opening 838, terminating in a first end within the remaining fluid at or substantially at a bottom portion 842 of the container 802. In some embodiments, the water supply tubing 836 may terminate at the opening 838. For example, when the opening 838 is at or adjacent to the bottom portion 822 of the container 802 a dip tube may not be required. A lumen extends through the water supply tubing 836 for receiving a flow of fluid therethrough. The lumen of the lens wash supply/irrigation supply tubing 836 is in selective operative fluid communication with the bottom portion of the container 802. In the illustrated embodiment, the gas supply tubing 834 and the water supply tubing 836 may enter the container 802 through a single or common opening 838. For example, the gas supply tubing 834 and the water supply tubing 836 may be coaxially arranged. However, this is not required. In some cases, the gas supply tubing 834 and the water supply tubing 836 may extend in a side by side arrangement. The opening 838 may include a grommet or heat seal 844 configured to seal the container 802 about the tubing 834, 836 in a fluid and pressure tight manner.

A portion of a gas supply tubing 834 and a portion of lens wash supply tubing 836 may extend from the reservoir 800, respectively, and may be connected in fluid communication with the endoscope at gas/lens wash connection on the connector portion 265 of the umbilical. The gas supply tubing 834 is connected in fluid communication with a gas pump (not explicitly shown) and gas feed line (not explicitly shown), and the lens wash supply tubing 836 is connected in fluid communication with lens wash feed line (not explicitly shown), within connector portion 265. While not explicitly shown, irrigation supply tubing may be coupled to the water supply tubing 836 via a manifold to supply irrigation fluid from the reservoir 800 or a separate irrigation supply tubing may be provided.

It is contemplated that the reservoir 800 may be filled and refilled as needed by removing the cap 808 and pouring water into the first receptacle 804. The refilling of the reservoir 800 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 800 with sterile or non-sterile water may create more flexibility and reduce the need to have as much sterile water in storage. Further, refilling the reservoir 800 via the port 806 and removable cap 808 may also remove the need to disconnect the reservoir 800 from the tubing 834, 836 throughout the day eliminating or greatly reducing the possibility of cross contamination by removing the need to replace the water container.

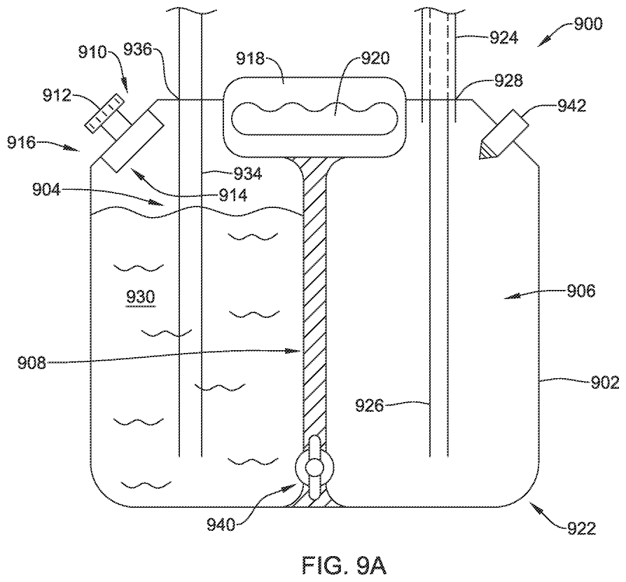

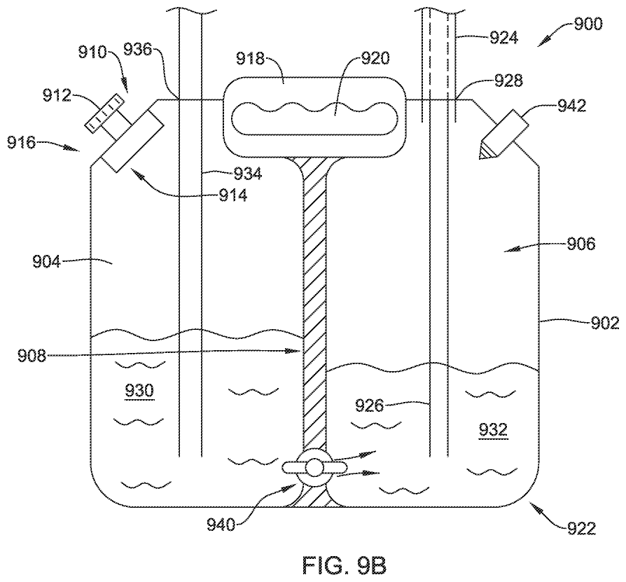

FIG. 9A depicts a side view of an illustrative refillable fluid reservoir 900 in a first configuration and FIG. 9B depicts the illustrative refillable fluid reservoir 900 in a second configuration. The reservoir 900 may be configured to be used in an endoscopic system and includes components similar to the endoscope and endoscope systems described with regard to FIGS. 1-4; however, not all features may be described or shown here if not pertinent to the fluid circuit of the system. The reservoir 900 includes a receptacle or container 902 defining a first receptacle or chamber 904 and a second receptacle or chamber 906. The first and second chambers 904, 906 may be fluidly isolated from one another via a seal 908 which extends from the top portion 916 to the bottom portion 922 of the container 902. The seal 908 may be permanent (e.g., not intended to open and close) or reversible (e.g., intended to open or close). For example, a permanent seal 908 may be provided by heat or pressure welding the container 902 along the seal 908. This is just one example. Other methods of creating a seal may be used as desired. In another example, waterproof hook and loop closures or zip-style closures may be used to provide a reversible seal 908.

The container 902 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In some embodiments, the container 902 may be entirely translucent, entirely opaque, or combinations thereof. The reservoir 900 may further include a port 910 having a removable cap 912. The port 910 may be positioned adjacent a top portion 916 of the container 902. The cap 912 may be formed from more rigid material (relative to the container 902) and may be configured to form a fluid tight seal with the port 910. The cap 912 may be configured to threadably engage the port 910, form a friction fit with the port 910, form a snap fit with the port 910 or otherwise releasably engage the port 910. In some examples, the port 910 and/or cap 912 may be formed from polyethylene terephthalate (PET), polypropylene (PP), etc. Portions of the port 910 may extend into the first chamber 904. The removable cap 912 may be removed to place a fluid source in selective fluid communication with the first chamber 904 and allow fluid to be poured through a lumen 914 of the port 910 and into the first chamber 904. Alternatively, or additionally a port may be in fluid communication with the second chamber 906. A stopcock or other actuatable valve 940 may be molded or otherwise provided within the seal 908, or otherwise disposed between the first and second chambers 904, 906, such that fluid 930 from the first chamber 904 may be transferred to the second chamber 906. Referring to FIG. 9B, opening the stopcock 940 creates a fluid path between the first chamber 904 and the second chamber 906 such that fluid 930 from the first chamber 904 enters the second chamber 906. The stopcock 940 may be closed when the desired volume of fluid 932 is in the second chamber 906.

Generally, the first and second chambers 904, 906 may provide separate fluid sources for lens wash and irrigation. For example, the first chamber 904 may provide fluid for irrigation while the second chamber 906 may provide fluid for lens wash. Having two separate chambers or chambers 904, 906 may improve the time required to pressurize the container 902 to enable lens washing capability. For example, if fluid for both lens wash and irrigation are drawn from the same source or container, the entire container must be pressurized to utilize the lens wash which may cause a lag between the input to lens wash and the function of actually washing the lens. It is contemplated that the first chamber 904 may be utilized for irrigation while the second chamber 906 may use utilized for insufflation and lens wash. The reverse configuration is also contemplated. In some embodiments, a pressure relief valve 942 may be molded into or otherwise provided in the second chamber 906. While not explicitly shown, a pressure relief valve or vent may be additionally or alternatively provided in the first chamber 904.

The reservoir 900 may include a carrying handle or other carrying mechanism 918. The handle 918 may define an opening or through hole 920 for receiving a hand or hook therethrough to carry the reservoir 900. In some cases, the carrying handle 918 may include an ergonomic grip for the user. It is contemplated that the handle 918 may be formed from a similar material as the cap 912 or from a same material as the container 902. In some examples, the handle may be formed from polyethylene terephthalate (PET), polypropylene (PP), etc.

The reservoir 900 may be movable between a collapsed storage configuration (not explicitly shown) and an expanded use configuration (FIG. 9A). In the use configuration, the bottom portion 922 may have a width that allows the reservoir 900 to remain upright without user intervention (e.g., with the port 910 and cap 912 upwards). The bottom portion 822 and or sides may include folds or pleats that allow the container 902 to fold or collapse.

In the collapsed storage configuration, the top portion 916 and the bottom portion 922 may have a similar width which allows the reservoir 900 to lay substantially flat such that the reservoir 900 may be stacked with other fluid reservoirs 900. In other examples, the reservoir 900 may be folded to reduce the amount of storage space it occupies. In some cases, since the reservoir 900 is sealed or capable of being sealed, a vacuum may be pulled during packaging of the reservoir 900 to further reduce the storage space required to store the reservoir 900. In some embodiments (e.g., in the case of a reversible seal), the reservoir 900 may be stored inside out such that the tubing 924, 926, 934 fittings and/or other components are within the first and/or second chamber 904, 906. The reservoir 900 may be stored in sterile packaging to maintain sterility of the tubing 924, 926, 934 and interior surface of the first and second chambers 904, 906. In some embodiments, the tubing 924, 934 may be stored within a second receptacle (not shown) as previously described.

The reservoir 900 may be connected in fluid communication with a gas supply/alternate gas supply tubing (or gas supply tubing) 924 and a lens wash supply/irrigation supply tubing 926. The gas supply tubing 924 extends from a second end external to the reservoir 900 through a reservoir opening 928 adjacent the top portion 916 of the second chamber 906. The shared gas supply tubing 924 may terminate within a reservoir gap, at or below the opening 928, but not extending into the remaining fluid in the second chamber 906. However, in some cases, the gas supply tubing 924 may extend into the fluid. For example, the opening 928 may be at a bottom or side of the container 902 such that the shared gas supply tubing 924 terminates within the fluid with gas bubbling up through the fluid 932 to pressurize the second chamber 906 (or both the first and second chambers 904, 906). A lumen extends through the gas supply tubing 924 for receiving a flow of air and/or gas therethrough. The lumen of the gas supply tubing 924 is in operative fluid communication with a top portion 916 of the second chamber 906. The water supply tubing 926 extends from a second end external to the reservoir 900 through the reservoir opening 928, terminating in a first end within the remaining fluid 932 at or substantially at a bottom portion 922 of the container 902. In some embodiments, the water supply tubing 926 may terminate at the opening 928. For example, when the opening 928 is at or adjacent to the bottom portion 922 of the container 902 a dip tube may not be required. A lumen extends through the water supply tubing 926 for receiving a flow of fluid therethrough. The lumen of the lens wash supply/irrigation supply tubing 926 is in selective operative fluid communication with the bottom portion of the second chamber 906. In the illustrated embodiment, the gas supply tubing 924 and the water supply tubing 926 may enter the container 902 through a single or common opening 928. For example, the gas supply tubing 924 and the water supply tubing 926 may be coaxially arranged. However, this is not required. In some cases, the gas supply tubing 924 and the water supply tubing 926 may extend in a side by side arrangement or may be separately connected to the container 902 in different locations. The opening 928 may include a grommet or heat seal configured to seal the container 902 about the tubing 924, 926 in a fluid and pressure tight manner.

In some embodiments, the reservoir 900 may be connected in fluid communication with a lumen of an irrigation supply tube 934. The irrigation supply tubing 934 extends from a second end to a first end which extends through an opening 936 and into an interior of the first chamber 904. In the use configuration, the second end of the irrigation supply tubing 934 may be external to the reservoir 900. A lumen extends through the irrigation supply tube 934 for receiving a flow of fluid therethrough. In some cases, the irrigation supply tube 934 may be coupled to a manifold, if so provided. The first end of the irrigation supply tube 934 is in selective fluid communication with the bottom portion 922 of the first chamber 904. The opening 936 may include a grommet or heat seal configured to seal the container 902 about the tubing 934 in a fluid and pressure tight manner.

The second ends of the gas supply tubing 924 and the lens wash supply tubing 926 may be connected in fluid communication with the endoscope at gas/lens wash connection on the connector portion 265 of the umbilical. The gas supply tubing 924 is connected in fluid communication with a gas pump (not explicitly shown) and gas feed line (not explicitly shown), and the lens wash supply tubing 926 is connected in fluid communication with lens wash feed line (not explicitly shown), within connector portion 265. The irrigation tubing 934 is connected in fluid communication with the irrigation supply line 255c via an irrigation pump 315.

It is contemplated that the reservoir 900 may be filled and refilled as needed by removing the cap 912 and pouring water into the first chamber 904. Further, the stopcock 940 may be opened and closed as necessary to transfer fluid from the first chamber 904 to the second chamber 906. The refilling of the reservoir 900 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 900 with sterile or non-sterile water may create more flexibility and reduce the need to have as much sterile water in storage. Further, refilling the reservoir 900 via the port 910 and removable cap 912 may also remove the need to disconnect the reservoir 900 from the tubing 924, 926, 934 throughout the day eliminating or greatly reducing the possibility of cross contamination by removing the need to replace the water container.

When the seal 908 is reversible, the container 902 may be filled and/or refilled with the seal 908 either in the open (non-bonded) or closed (bonded) configuration. When the seal 908 is in the open configuration, the user may fill the container through the port 910 with fluid entering both the first and second chambers 904, 906. When the container 902 is filled as desired, the seal 908 may then be closed (e.g., pushing the hook and loop together or pushing the zip style closure together) thus creating the two chambers 904, 906. The opening and closing of the seal 908 may be configured to occur as a one-time use or may be repeatable. When the seal 908 is in the closed configuration, the user may pull on the outside of the reservoir 900 to open the seal 908 and join the two chambers 904, 906 into a common chamber and then fill to the desired level. After filling the user may then close the seal 908 to create two separate chambers 904, 906 once again. It is contemplated that the exterior of the reservoir 900 may be provided with a grip or grasping mechanism to facilitate opening and closing of the seal 908.

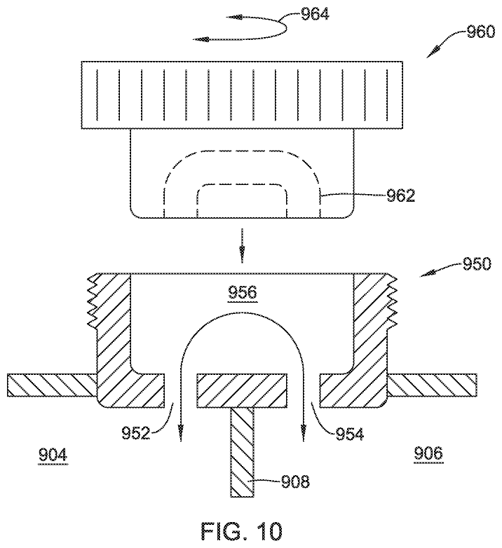

FIG. 10 is partial cross-sectional view of an alternative fill port 950 for a dual chamber reservoir, such as reservoir 900. The port 950 may be positioned adjacent the seal 908 such that the port 950 spans the seal 908. The port 950 may include a cavity 956 for receiving fluid from an external source. The port 950 may further include a first opening 952 configured to fluidly couple the cavity 956 with the first chamber 904 and a second opening 954 configured to fluidly couple the cavity 956 with the second chamber 906. Fluid may enter the cavity 956 and fill the first and second chambers 904, 906 simultaneously via the openings 952, 954.

A removable cap 960 may be secured to the port 950 to close the opening 952, 954 and provide two separate chambers 904, 906. The cap 960 may be configured to threadably engage the port 950, form a friction fit with the port 950, form a snap fit with the port 950 or otherwise releasably engage the port 950. The cap 960 may further include a generally “U”-shaped channel 962. The channel 962 may provide an internal flow path between the first and second chambers 904, 906 such that fluid may be moved between the first and second chambers 904, 906, as needed. It is contemplated that the cap 960 may be rotated, as shown at arrow 964, to selectively fluidly couple/uncouple the channel 962 with the openings 952, 954. The cap 960 may include visual indicia to allow the user to readily discern whether the cap 960 is rotated to fluidly seal the openings 952, 954 or to fluidly couple the openings 952, 954. In some embodiments, the internal flow path 962 may include a check valve configured to control a direction of fluid flow (e.g., only allow fluid flow in one direction) or to maintain a differential internal pressure.

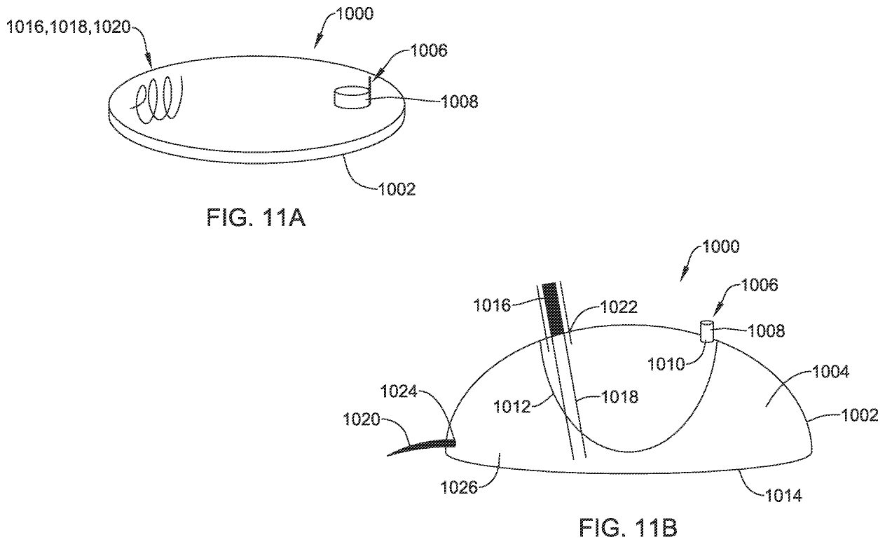

FIG. 11A depicts a perspective view of an illustrative refillable fluid reservoir 1000 in a storage configuration and FIG. 11B depicts the refillable fluid reservoir 1000 in a use configuration. The reservoir 1000 may be configured to be used in an endoscopic system and includes components similar to the endoscope and endoscope systems described with regard to FIGS. 1-4; however, not all features may be described or shown here if not pertinent to the fluid circuit of the system. The reservoir 1000 includes a container 1002 defining a first receptacle 1004. The container 1002 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In some embodiments, the container 1002 may be entirely translucent, entirely opaque, or combinations thereof. The reservoir 1000 may further include a port 1006 having a removable cap 1008. The cap 1008 may be formed from more rigid material (relative to the container 1002) and may be configured to form a fluid tight seal with the port 1006. The cap 1008 may be configured to threadably engage the port 1006, form a friction fit with the port 1006, form a snap fit with the port 1006 or otherwise releasably engage the port 1006. In some examples, the port 1006 and/or cap 1008 may be formed from polyethylene terephthalate (PET), polypropylene (PP), etc. Portions of the port 1006 may extend into the first receptacle 1004. The removable cap 1008 may be removed to place a fluid source in selective fluid communication with the first receptacle 1004 and allow fluid to be poured through a lumen 1010 of the port 1006 and into the first receptacle 1004.

The container 1002 may include an expandable frame or support ring 1012. The frame 1012 may be incorporated or embedded into the container 1002 or attached to an inner or outer surface thereof. The frame 1012 can be made from a number of different materials such as, but not limited to, metals, metal alloys, shape memory alloys and/or polymers, as desired, enabling the frame 1012 to be expanded into shape when the reservoir 1000 is in use. Depending on the material selected for construction, the frame 1012 may be self-expanding (i.e., configured to automatically expand when unconstrained). As used herein the term “self-expanding” refers to the tendency of the frame 1012 to return to a preprogrammed shape when unrestrained from an external biasing force (for example, but not limited to, a storage bag or container, a strap, etc.). When in the storage or collapsed configuration (FIG. 11A), the frame 1012 may be biased into a folded configuration which allows the container 1002 to be folded upon itself reducing the volume thereof. In the collapsed configuration, the frame 1012 may lie in a first plane. A biasing or external force may be applied to the reservoir 1000 to maintain the frame 1012, and thus the container 1002, in the collapsed configuration.

In the collapsed storage configuration, the reservoir 1000 to lay substantially flat such that the reservoir 1000 may be stacked with other fluid reservoirs 1000. In other examples, the reservoir 1000 may be rolled or folded to reduce the amount of storage space it occupies. In some cases, since the reservoir 1000 is sealed or capable of being sealed, a vacuum may be pulled during packaging of the reservoir 1000 to further reduce the storage space required to store the reservoir 1000.

When it is desired to be used, the biasing force may be removed from the reservoir 1000 and the frame 1012 resumes is “preprogrammed” shape (i.e., a shape to which frame 1012 is biased when the biasing force is absent/a shape-memory shape of frame 1012). In the unbiased configuration, the frame 1012 may generally take the shape of a saddle or a curved ellipse. The frame 1012 may take other shapes as desired, such as, but not limited to, circular, square, rectangular, polygonal, etc. In some examples, the container 1002 may have a generally hemispherical or dome shape in the use configuration. However, this is not required. The container 1002 may take other shapes, as desired. In the expanded or use configuration, the frame 1012 may move to a different plane than the collapsed configuration for providing opening of stiffening to the container 1002 by adding structure in a plane that was previously collapsed. In the expanded configuration, the cap 1008 may be removed and air allowed to enter the first receptacle 1004 to help facilitate expansion of the container 1002. When expanded, the container 1002 may have sufficient rigidity to stand on its own. In some examples, the bottom portion 1014 of the container 1002 may be generally flat to provide a base that allows the reservoir 1000 to remain in an upright configuration.

While not explicitly shown, the reservoir 1000 may include a handle or other carrying feature. The handle may define an opening or through hole for receiving a hand therethrough to carry the reservoir 1000. In some cases, the carrying handle may include an ergonomic grip for the user. It is contemplated that the handle may be formed from a similar material as the cap 1008 or may be formed from the same material as the container 1002. In some examples, the handle may be formed from polyethylene terephthalate (PET), polypropylene (PP), etc.

The reservoir 1000 may be connected in fluid communication with a lumen of a gas supply tube 1016 and a lumen of a water supply tube 1018. The gas supply tube 1016 and the water supply tube 1018 may be coaxially arranged with the water supply tube 1018 extending within and through the lumen of the gas supply tube 1016 along a portion of the length of the gas supply tube 1016. However, this is not required. In some cases, the gas supply tube 1016 and the water supply tube 1018 may extend side by side. The gas supply tubing 1016 extends from a second end to a first end adjacent to an opening 1022 in the first receptacle 1004. In the use configuration, the second end of the gas supply tubing 1016 may be external to the reservoir 1000. In the storage configuration, the second end of the gas supply tubing 1016 may be stored in the first receptacle 1004 or folded along an exterior of the container 1002. A lumen extends through the gas supply tube 1016 for receiving a flow of air and/or gas therethrough. The lumen of the gas supply tube 1016 is in fluid communication with the first receptacle 1004. The water supply tubing 1018 extends from a second end to a first end which extends through the opening 1022 and into an interior of the first receptacle 1004. In the use configuration, the second end of the water supply tubing 1018 may be external to the reservoir 1000. In the storage configuration, the second end of the water supply tubing 1018 may be stored in the first receptacle 1004 or folded along an exterior of the container 1002. A lumen extends through the water supply tube 1018 for receiving a flow of fluid therethrough. The second ends of the gas supply tube 1016 and the water supply tube 1018 may be coupled to a manifold (if so provided) or a connector portion 265 of an endoscope system. The first end of the water supply tube 1018 is in selective fluid communication with the bottom portion 1026 of the first receptacle 1004. The opening 1022 may include a grommet or heat seal configured to seal the container 1002 about the tubing 1016, 1018 in a fluid and pressure tight manner.

In some embodiments, the reservoir 1000 may be connected in fluid communication with a lumen of an irrigation supply tube 1020. The irrigation supply tubing 1020 extends from a second end to a first end which extends through an opening 1024 and into an interior of the first receptacle 1004. In the use configuration, the second end of the irrigation supply tubing 1020 may be external to the reservoir 1000. In the storage configuration, the second end of the irrigation supply tubing 1020 may be stored in the first receptacle 1004 or folded along an exterior of the container 1002. A lumen extends through the irrigation supply tube 1020 for receiving a flow of fluid therethrough. In some cases, the irrigation supply tube 1020 may be coupled to a manifold, if so provided. The first end of the irrigation supply tube 1020 is in selective fluid communication with the bottom portion 1026 of the first receptacle 1004. The opening 1024 may include a grommet or heat seal configured to seal the container 1002 about the tubing 1020 in a fluid and pressure tight manner.

The second ends of the gas supply tubing 1016 and the lens wash supply tubing 1018 may be connected in fluid communication with the endoscope at gas/lens wash connection on the connector portion 265 of the umbilical. The gas supply tubing 1016 is connected in fluid communication with a gas pump (not explicitly shown) and gas feed line (not explicitly shown), and the lens wash supply tubing 1018 is connected in fluid communication with lens wash feed line (not explicitly shown), within connector portion 265. The irrigation tubing 1020 is connected in fluid communication with the irrigation supply line 255c via an irrigation pump 315.

To use the reservoir 1000, the biasing force may be removed from the reservoir 1000 to allow the container 1002 to expand. The cap 1008 may be temporarily removed from the port 1006 to allow air to enter the first receptacle 1004. Thus, when lens wash is desired, there may be enough air already in the first receptacle 1004 to keep the receptacle 1004 primed. The cap 1008 may be reattached to the port 1006 before use of the reservoir 1000. The tubing 1016, 1018, 1020 may be removed from the storage configuration and coupled to the endoscope system. In some embodiments, the tubing 1016, 1018, 1020 may be provided within a second receptacle (not shown) in the storage configuration, as previously described. The first receptacle 1004 may be filled with sterile or non-sterile water via the port 1006. In the use configuration, the bottom portion 1026 may have a width that allows the reservoir 1000 to remain upright without user intervention.

It is contemplated that the reservoir 1000 may be filled and refilled as needed by removing the cap 1008 and pouring water into the first receptacle 1004. The refilling of the reservoir 1000 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 1000 with sterile or non-sterile water may create more flexibility and reduce the need to have as much sterile water in storage. Further, refilling the reservoir 1000 via the port 1006 and removable cap 1008 may also remove the need to disconnect the reservoir 1000 from the tubing 1016, 1018, 1020 throughout the day eliminating or greatly reducing the possibility of cross contamination by removing the need to replace the water container.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container arranged and configured to couple to an endoscope for use in an endoscopic procedure, the container comprising:
a flexible container configured to contain a fluid within a first receptacle thereof, the container having a bottom portion and a top portion and movable between a collapsed storage configuration and an expanded use configuration;
a water supply tube including a first end, a second end, and a first lumen extending therethrough, wherein the first lumen is in fluid communication with the first receptacle in the bottom portion of the container and the second end of the water supply tube is positioned external to the container;
a gas supply tube including a first end, a second end, and a second lumen extending therethrough, wherein the second lumen is in operative fluid communication with the first receptacle and the second end of the gas supply tube is positioned external to the container;
a port positioned adjacent to the top portion of the container, wherein the port is configured to selectively fluidly couple the first receptacle of the container with an external water source; and
a removable cap selectively coupled to the port.

2. The container of claim 1, further comprising a carrying handle adjacent the top portion of the container.

3. The container of claim 1, further comprising a second receptacle, the second receptacle configured to store the water supply tube and the gas supply tube when the container is the collapsed storage configuration.

4. The container of claim 3, wherein the second receptacle is sealed prior to use of the container and unsealed at a time of use of the container.

5. The container of claim 1, wherein when in the expanded use configuration, the bottom portion of the container is configured to support the container in an upright configuration.

6. A container arranged and configured to couple to an endoscope for use in an endoscopic procedure, the container comprising:
a container configured to contain a fluid within a first receptacle thereof, the container having a bottom portion and a top portion and movable between a collapsed storage configuration and an expanded use configuration;
a water supply tube including a first end, a second end, and a first lumen extending therethrough, wherein the first lumen is in fluid communication with the first receptacle in the bottom portion of the container and the second end of the water supply tube is positioned external to the container;
a gas supply tube including a first end, a second end, and a second lumen extending therethrough, wherein the second lumen is in operative fluid communication with the first receptacle and the second end of the gas supply tube is positioned external to the container;
a port positioned adjacent to the top portion of the container, wherein the port is configured to selectively fluidly couple the first receptacle of the container with an external water source; and
a support structure.

7. The container of claim 6, wherein in an absence of a biasing force, the support structure is configured to maintain the container in the expanded use configuration.

8. The container of claim 6, wherein the support structure comprises a support ring or pole.

9. The container of claim 6, wherein the support structure comprises a planar reinforcing member movable from a first configuration to a second configuration.

10. The container of claim 9, wherein when the planar reinforcing member is in the second configuration, the support structure is generally parallel to a bottom surface of the container.

11. The container of claim 9, wherein when the planar reinforcing member is in a first configuration the container is in the collapsed storage configuration and when the planar reinforcing member is in the second configuration the container is in the expanded use configuration.

12. The container of claim 6, wherein the support structure extends from a first corner of the container adjacent the top portion thereof to a second corner of the container adjacent the bottom portion of the container.

13. The container of claim 12, wherein the first corner and the second corner are at opposing lateral sides of the container.

14. A container arranged and configured to couple to an endoscope for use in an endoscopic procedure, the container comprising:
a container configured to contain a fluid therein, the container having a bottom portion and a top portion and movable between a collapsed storage configuration and an expanded use configuration;
a water supply tube including a first end, a second end, and a first lumen extending therethrough, wherein the first lumen is in fluid communication with the bottom portion of the container and the second end of the water supply tube is positioned external to the container;
a gas supply tube including a first end, a second end, and a second lumen extending therethrough, wherein the second lumen is in operative fluid communication with container and the second end of the gas supply tube is positioned external to the container;
a port positioned adjacent to the top portion of the container, wherein the port is configured to selectively fluidly couple the first receptacle of the container with an external water source; and
a seal extending from the top portion of the container to the bottom portion of the container, the seal configured to divide the container into a first chamber and a second chamber.

15. The container of claim 14, further comprising an actuatable valve disposed between the first chamber and the second chamber.

16. The container of claim 14, further comprising a pressure relief valve in fluid communication with the second chamber.

17. The container of claim 14, wherein the port is in fluid communication with the first chamber and the second chamber.

18. The container of claim 17, further comprising a cap configured to be releasably coupled to the port, the cap configured to selectively fluidly couple the first and second chambers.

19. The container of claim 18, wherein the seal is selectively opened and closed, wherein the when the seal is open the first and second chambers are fluidly coupled and when the seal is closed the first and second chambers are fluidly isolated from one another.

20. The container of claim 18, wherein the seal comprises a fluid-tight hook and loop fastener.
